(19) United States
(12) Patent Application Publication
(10) Pub. No.: US 2019/0209209 A1
(43) Pub. Date: Jul. 11, 2019

(54) METHOD AND DEVICE FOR INSERTING AT LEAST ONE MEDICAL COMPONENT WITHIN THE BODY

(71) Applicant: Mark A. Wendel, Jonesboro, AR (US)

(72) Inventor: Mark A. Wendel, Jonesboro, AR (US)

(21) Appl. No.: 16/246,210

(22) Filed: Jan. 11, 2019

Related U.S. Application Data
(63) Continuation-in-part of application No. 62/616,358, filed on Jan. 11, 2018.

(51) Int. Cl. A61B 17/34

(52) U.S. Cl. CPC A61B 17/3468; A61B 17/3401; A61M 2025/0007; A61B 2017/3445; A61B 17/3421

(57) ABSTRACT

The dilator catheter accepts at least one or more medical components such as electrically conductive leads, wires, or catheter for a medical device and/or procedure. The dilator catheter provides an inner sleeve and an outer sleeve. The inner sleeve inserts into an opening of the outer sleeve. The opening of the outer sleeve is sized such that the inner sleeve will contact the outer sleeve to secure the inner sleeve within the outer sleeve. The inner sleeve and the outer sleeve provide an opening that extends throughout the dilator catheter. The user places the distal end of the dilator catheter at the desired location for introducing the medical component. The user then removes the inner sleeve to enlarge the opening. The user then directs the medical component(s) through the opening to the distal end of the dilator catheter for proper placement of the medical component(s).

### CROSS-REFERENCE TO RELATED APPLICATIONS

This application is a continuation in part of U.S. Patent Application No. 62/616,358 filed on Jan. 11, 2018 entitled METHOD AND DEVICE FOR INSERTING MULTIPLE LEADS WITHIN THE BODY which is hereby incorporated by reference.

### STATEMENT REGARDING FEDERALLY SPONSORED RESEARCH OR DEVELOPMENT

Not Applicable.

### REFERENCE TO A MICROFICHE APPENDIX

Not Applicable.

### RESERVATION OF RIGHTS

A portion of the disclosure of this patent document contains material which is subject to intellectual property rights such as but not limited to copyright, trademark, and/or trade dress protection. The owner has no objection to the facsimile reproduction by anyone of the patent document or the patent disclosure as it appears in the Patent and Trademark Office patent files or records but otherwise reserves all rights whatsoever.

### BACKGROUND OF THE INVENTION

### 1. Field of the Invention

The invention relates to a technique and system for inserting at least one medical component, including but not limited to lead(s), wire(s), catheter(s), medicine, or fluid(s) into the body. The present invention relates to an epidural dilator catheter. More specifically, the present invention is related to inserting stimulator leads or wires within the epidural space. The present invention also applies to any medical devices and/or procedures that require implantation of a medical component, including but not limited to wires, catheters, electrically conductive leads, including pain management devices, neurologic stimulators or modulators, pacemakers, defibrillators or other medical devices providing rhythm management, resynchronization therapy or other cardiac therapy.

### II. Description of the Known Art

Patents, patent applications, and references disclosing relevant information are disclosed below. These patents, patent applications, and references are hereby expressly incorporated by reference in their entirety.

U.S. Pat. No. 9,849,279 issued to Tobin on Dec. 26, 2017 (“the '279 patent”) teaches a medical apparatus that includes an electrically conductive lead for a medical device, the lead having an internal bore terminating at a distal lead opening, and a lead delivery device for delivering the distal end of the lead to a blood vessel during implantation of the lead. The lead delivery device taught by the '279 patent includes a removably anchorable guidewire, and a fixator attached to a distal portion of the guidewire for anchoring the guidewire. The fixator taught by the '279 patent is movable between a compact configuration and an expanded configuration. The fixator taught by the '279 patent is capable of passing through the distal lead opening of the lead in the compact configuration. The fixator taught by the '279 patent is capable of exerting a holding force in the range of about 0.89 to 4.45 N in the lumen of the blood vessel in the expanded configuration.

U.S. Publication No. 20160302827 to Chitre on Oct. 20, 2016 (“the '827 publication”) teaches insertion devices and associated systems and methods for the percutaneous placement of patient leads. A system taught by the '827 publication teaches a cannula having a lumen and a first dilator. The first dilator taught by the '827 publication can be positioned within the lumen and the first dilator and the cannula can be used to create a percutaneous entry point. An additional dilator taught by the '827 publication can be positioned over the first dilator and advanced into the percutaneous entry point to expand the percutaneous entry point. A final dilator taught by the '827 publication can be inserted into the patient and two leads can be advanced into the patient through the final dilator.

### SUMMARY OF THE INVENTION

The present invention provides a dilator catheter as an introducer conduit that accepts a medical component for insertion within a patient. Such medical components may include, but are not limited to, wires, catheters, electrically conductive leads, including pain management devices, neurologic stimulators or modulators, pacemakers, defibrillators or other medical components providing rhythm management, resynchronization therapy or other cardiac therapy. The dilator catheter provides an inner sleeve and an outer sleeve. The inner sleeve attaches interior of the outer sleeve. The inner sleeve fits into an opening of the outer sleeve. The inner sleeve also provides an opening. The opening of the inner sleeve is smaller than the opening of the outer sleeve.

The introducer conduit may accept catheter(s), wire(s), or lead(s) for implantation within a patient. The leads attach to a medical device, including but not limited to a spinal cord stimulation system, pacemaker, or other medical device that requires implantation of electrically conductive leads, including neurologic modulators, pacemakers, defibrillators or other medical devices providing electrical current, rhythm management, resynchronization therapy or other cardiac therapy.

The present invention assists with placement of the leads into the body. A user inserts a needle into the body. The user places the needle into the body directing the needle to the area in which the leads are to be placed.

In one embodiment, the user then places the needle into the location at which the conductors are to be applied, such as the epidural space. The user inserts a wire though the needle and directs the wire into the location at which the conductors are to be applied. In one embodiment, the user directs the wire into the epidural space and in the cranial direction through the epidural space.

The user then places the opening of the inner sleeve of the dilator catheter onto the wire. The user guides the dilator catheter to the desired location via the wire.

After placing the dilator catheter into the proper position, the user removes the wire through the inner sleeve. The user also removes the inner sleeve from the outer sleeve to increase the size of opening 106 to the size of opening 110. The outer sleeve of the dilator catheter remains in position to maintain opening 110.

The outer sleeve provides an opening that accepts a catheter, at least one or more leads, or at least one or more wires. In one embodiment, the outer sleeve provides a double lumen opening. In other embodiments, the opening within the outer sleeve accepts one or more leads or wires, including two or more leads or wires. Such an outer sleeve accepts at least one or more leads or wires for placement within the patient.

Further areas of applicability of the present invention will become apparent from the description provided hereinafter. It should be understood that the description and specific examples are intended for purposes of illustration only and are not intended to limit the scope of the present teachings.

Accordingly, it is an object of the present invention to provide a dilator catheter for applying conductive leads within the patient's body.

It is another object of the present invention to provide an introducer conduit that accepts one or more medical components for insertion within a patient.

It is another object of the present invention to simplify the process of installing the leads.

It is another object of the present invention to reduce the time required to install the leads within the patient.

It is another object of the present invention to reduce trauma to the patent when installing the leads.

It is another object of the present invention to reduce the number of times that a user is required to apply wires within a patient.

It is another object of the present invention to provide a single catheter that accepts at least one or more leads, catheters, or wires for applying within a patient.

It is another object of the present invention to install two leads using a single catheter.

These and other objects and advantages of the present invention, along with features of novelty appurtenant thereto, will appear or become apparent in the course of the following descriptive sections and the attached drawings.

### BRIEF DESCRIPTION OF THE DRAWINGS

In the following drawings, which form a part of the specification and which are to be construed in conjunction therewith, and in which like reference numerals have been employed throughout wherever possible to indicate like parts in the various views:

FIG. 1 is a perspective view of one embodiment of the present invention;

FIG. 2 is a sectional view of an inner sleeve of one embodiment of the present invention;

FIG. 3 is a sectional view of an outer sleeve of one embodiment of the present invention;

FIG. 4 is a sectional view of an inner sleeve of one embodiment of the present invention;

FIG. 5 is a sectional view of an outer sleeve of one embodiment of the present invention;

FIG. 6 is an environmental view of one embodiment of the present invention;

FIG. 7 is an environment view thereof; and

FIG. 8 is an environmental view thereof.

### DETAILED DESCRIPTION

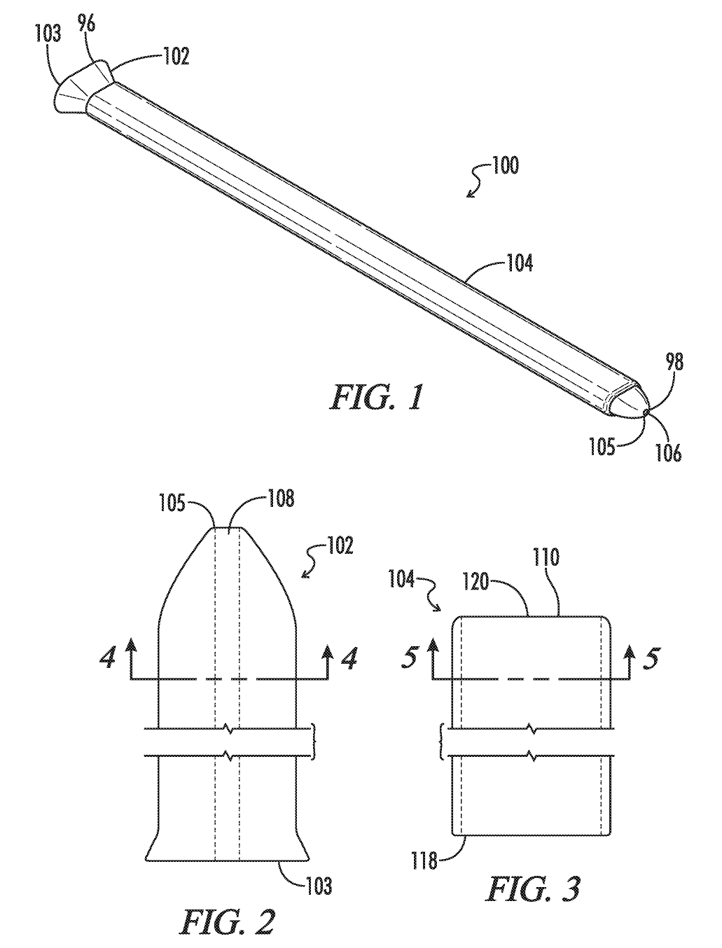

FIG. 1 shows the dilator catheter 100 of the present invention. The dilator catheter 100 accepts at least one or more medical components, including but not limited to electrically conductive leads, catheter(s), or at least one or more wires, medicine, or fluid(s) for a medical device and/or procedure. The dilator catheter provides an inner sleeve 102 and an outer sleeve 104. The inner sleeve 102 inserts into the interior of the outer sleeve 104. The fit of the inner sleeve 102 within the outer sleeve 104 secures the inner sleeve 102 within the outer sleeve 104. The inner sleeve 102 and the outer sleeve 104 provide an opening 106 that extends throughout the dilator catheter 100. The opening 106 extends from the proximal end 96 of the dilator catheter to the distal end 98 of the dilator catheter.

The inner sleeve 102 provides a flared end 103. The flared end 103 is located on the proximal end of the dilator catheter 100. The proximal end of the inner sleeve 102 increases in size. The increased size of the proximal end of the inner sleeve 102 prevents inner sleeve 102 from passing through the opening of the outer sleeve. The increased size of the proximal end of the inner sleeve 102 provides an increased surface area for the user to grip the inner sleeve 102 for removal of the inner sleeve 102 from the outer sleeve 104. The flared end 103 protrudes outward from the outer sleeve 104 at the proximal end.

In one embodiment, the distal end 105 of the inner sleeve 102 narrows to a tapered distal end 105. This narrowed distal end 105, such as a tapered end, of the inner sleeve 102 extends beyond the distal end of the outer sleeve 104 such that the inner sleeve 102 protrudes outward from the outer sleeve 104 at the distal end.

The user places the distal end 98 of the dilator catheter 100 at the desired location within the patient. The user then removes inner sleeve 102 from the outer sleeve 104. Removing the inner sleeve 102 increases the size of the opening 106 to opening 110 of the outer sleeve 104.

The flared end 103 increases a surface area of the proximal end. The increased surface area increases the area for the user to grip and remove the inner sleeve from the outer sleeve. The user grips flared end 103 and pulls the inner sleeve 102 from the outer sleeve 104. The outer sleeve 104 remains in place at the desired location.

Removing the inner sleeve 102 from the dilator catheter increases the size of opening 106. With the inner sleeve 102 removed from the dilator catheter 100, the size of the opening 106 increases for passage of a medical component including, but not limited to one or more leads or wires or a catheter through the outer sleeve 104 and opening 106. The user then directs the leads through opening 106, 110 to the distal end 98 of the dilator catheter 100 for proper placement of the leads.

FIG. 2 shows the inner sleeve 102, the flared end 103, and the tapered distal end 105. Opening 108 extends longitudinally through the inner sleeve 102. The opening 108 is located centrally of the inner sleeve 102 and extends from the distal end 105 at the narrow tapered end to the proximal end at which the flared end 103 is located. The opening 108 of inner sleeve 102 is smaller in size than the opening 110 of the outer sleeve 104.

The distal end of the inner sleeve provides a tapered end. The tapered distal end assists with insertion of the inner sleeve into the outer sleeve. The tapered end also reduces contact with the outer sleeve to ease the process of removing the inner sleeve from the outer sleeve.

FIG. 3 shows the outer sleeve 104. Similar to the inner sleeve 102, the opening 110 extends longitudinally through the outer sleeve 104. The opening 110 is located centrally of the outer sleeve 104 and extends from the distal end 120 to the proximal end 118.

The inner sleeve 102 is located within the outer sleeve 104. Opening 110 of the outer sleeve 104 accepts the inner sleeve 102. The opening 106 extends from the proximal end 96 of the dilator catheter 100 to the distal end 98 of the dilator catheter 100 of both the inner sleeve 102 and the outer sleeve 104.

The inner sleeve 102 fits within opening 106 of the outer sleeve 104. Removing inner sleeve 102 from the dilator catheter 100 increases the size of the opening 106 through the outer sleeve 104. The dilator catheter 100 with inner sleeve 102 removed provides sufficient space for one or more leads, or one or more wires, or a catheter to pass through the opening 110 of the outer sleeve 104. In one embodiment, the opening is sized for insertion of two or more wires or leads.

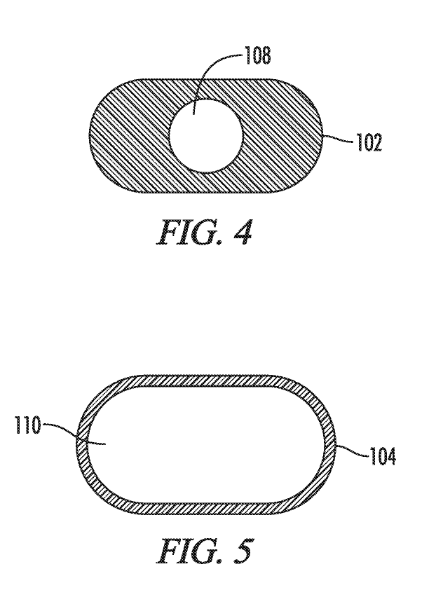

FIG. 4 shows a cross section of the inner sleeve 102. The inner sleeve 102 encompasses the opening 108. Opening 108 extends from the proximal end to the distal end of the inner sleeve 102.

FIG. 5 shows a cross section of the outer sleeve 104. The outer sleeve 104 encompasses the opening 110. Opening 110 extends from the proximal end to the distal end of the outer sleeve 104. Opening 110 is larger than opening 108. Opening 110 accepts the inner sleeve 102 within outer sleeve 104. Inner sleeve 102 inserts into opening 110 of the outer sleeve 104.

The inner sleeve 102 is sized slightly smaller than the opening 110 of the outer sleeve 104. The inner sleeve 102 contacts the inner surface of the outer sleeve 104. The inner sleeve 102 frictionally engages the outer sleeve to secure the inner sleeve 102 within the outer sleeve 104. The inner sleeve 102 is sized small enough to allow removal of the inner sleeve 102 from the outer sleeve 104.

The user removes the inner sleeve 102 from the outer sleeve 104 to enlarge opening 106 to the size of the opening 110. The enlarged opening 110 accepts the one or more components, including, but not limited to, one or more leads, including two or more leads, one or more wires, or one or more catheters. The medical component(s), such as conductors, can then pass through opening 110 to the location at which the component(s) are to be applied. The component(s) pass through opening 110 from the proximal end through the distal end.

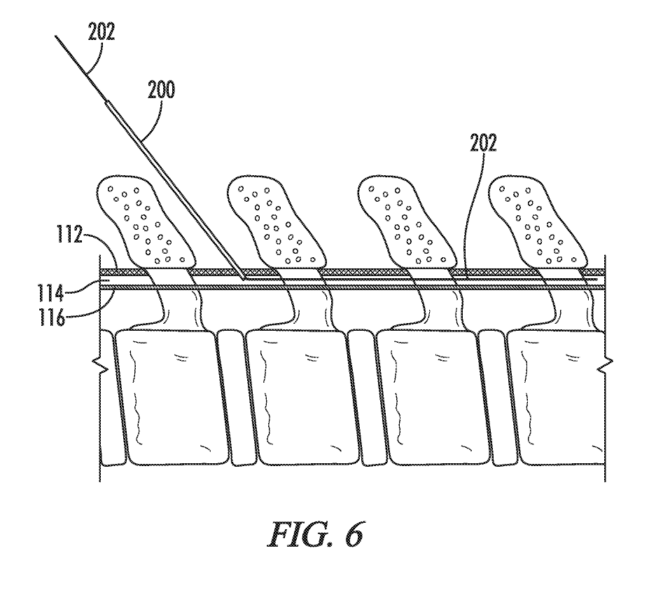

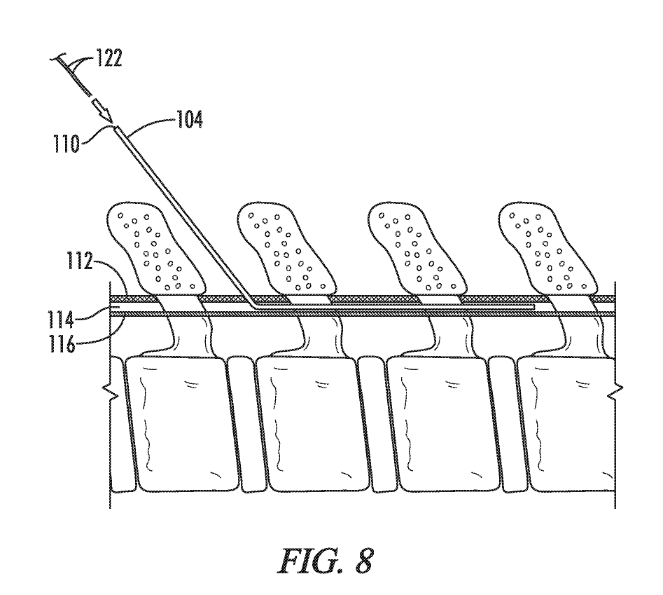

FIGS. 6-8 show use of the dilator catheter 100. The user inserts needle 200 into the epidural space 114 between the ligamentum flavum 112 and the dura 116. A guide wire 202 is then inserted through the needle 200 and into the epidural space 114. The user guides the distal end of the wire 202 to the desired location within the patient.

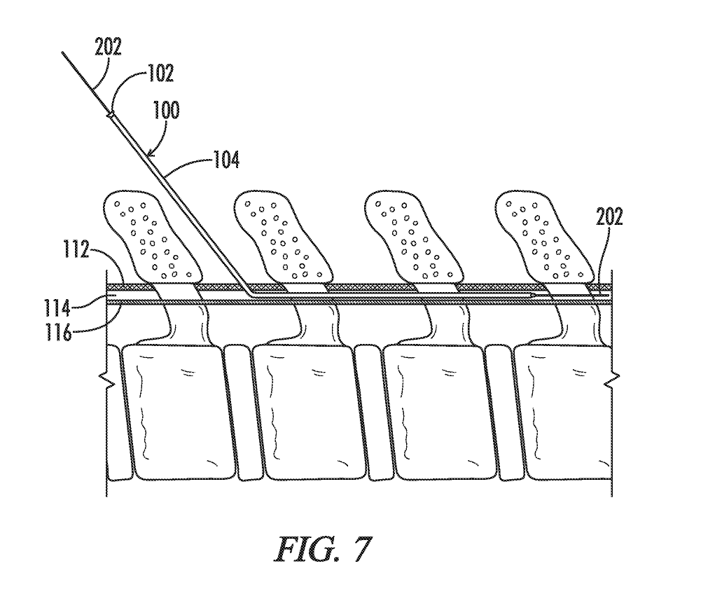

FIG. 7 shows the dilator catheter 100 inserted over the wire 202. The opening 106 of the dilator catheter 100 inserts over the wire 202. The user guides the distal end of the dilator catheter 100 to the desired location.

Once the dilator catheter 100 is placed in the proper position, the user removes the wire 202 and the inner sleeve 102. The outer sleeve 104 remains in position. The distal end of the outer sleeve 104 remains in the epidural space 114. Removing the inner sleeve 102 enlarges the opening of the dilator catheter 100 to accept the medical component which includes but is not limited at least one or more leads, such as two leads 122, at least one or more wires, or one or more catheters. The user then inserts the medical component, such as the one or more leads 122, the one or more wires, or the catheter into the opening 110 of the dilator catheter.

The outer surface of the dilator catheter provides a smooth outer surface. The outer sleeve and the inner sleeve provide a smooth transition along the dilator catheter. The tapered end of the inner sleeve narrows to a point for insertion into the patient.

While the device has been discussed as being implemented within the epidural space, the device may be implemented in other areas in which a medical component, including one or more leads, wires, or catheters may be required. The leads connect to a medical device, including but not limited to a spinal cord stimulation system, pacemaker, or other medical device that requires implantation of electrically conductive leads, including neurological modulators, pacemakers, defibrillators or other medical devices providing electrical current, rhythm management, resynchronization therapy or other cardiac therapy.

Another embodiment of the present invention provides ports on the outer sleeve. Such ports allow application of fluids, dyes, medicine, or material within the body to assist with procedures.

Further areas of applicability of the present invention will become apparent from the description provided hereinafter. It should be understood that the description and specific examples are intended for purposes of illustration only and are not intended to limit the scope of the present teachings.

From the foregoing, it will be seen that the present invention is one well adapted to obtain all the ends and objects herein set forth, together with other advantages which are inherent to the structure.

It will be understood that certain features and sub-combinations are of utility and may be employed without reference to other features and sub-combinations. This is contemplated by and is within the scope of the claims.

As many possible embodiments may be made of the invention without departing from the scope thereof, it is to be understood that all matter herein set forth or shown in the accompanying drawings is to be interpreted as illustrative and not in a limiting sense.

What is claimed is:

1. A dilator catheter device for inserting at least one or more medical components into a patient wherein the medical component passes longitudinally through the dilator catheter device, the device comprising:
an outer sleeve having a proximal end and a distal end;
an opening in the outer sleeve extending longitudinally from the proximal end to the distal end;
an inner sleeve inserted into the opening of the outer sleeve, the inner sleeve having a proximal end and a distal end, the inner sleeve having a greater length than the outer sleeve, wherein the inner sleeve protrudes longitudinally outward from the distal end of the outer sleeve and the inner sleeve protrudes longitudinally outward from the proximal end of the outer sleeve;
an opening in the inner sleeve extending longitudinally from the proximal end to the distal end.

2. The device of claim 1 wherein a cross section of the opening of the inner sleeve along the longitudinal axis is smaller than a cross section of the opening of the outer sleeve along the longitudinal axis.

3. The device of claim 1 further comprising:
a flared end of the inner sleeve located at the proximal end wherein the flared end is sized not to pass through the opening of the outer sleeve.

4. The device of claim 3 further comprising:
a tapered end of the inner sleeve located at the distal end.

5. The device of claim 4 wherein the inner sleeve is removable from the outer sleeve.

6. The device of claim 5 wherein the flared end of the inner sleeve is sized not to pass completely through the opening of the outer sleeve.

7. The device of claim 1 wherein the inner sleeve is flexible.

8. The device of claim 7 wherein the outer sleeve is flexible.

9. The device of claim 1 wherein the opening of the outer sleeve is sized to accept the insertion of at least two electrically conductive leads longitudinally through the opening of the outer sleeve from the proximal end to the distal end.

10. The device of claim 1 wherein the proximal end of the inner sleeve increases in size to limit passage of the inner sleeve through the outer sleeve.

11. A dilator catheter device for inserting at least one or more medical components into a patient wherein the component passes longitudinally through the dilator catheter device, the device comprising:
an outer sleeve having a proximal end and a distal end;
an opening in the outer sleeve extending longitudinally from the proximal end to the distal end;
an inner sleeve inserted into the opening of the outer sleeve, the inner sleeve having a proximal end and a distal end, the inner sleeve having a greater length than the outer sleeve, wherein the inner sleeve protrudes longitudinally outward from the distal end of the outer sleeve and the inner sleeve protrudes longitudinally outward from the proximal end of the outer sleeve, wherein the inner sleeve is removable from the outer sleeve;
an opening in the inner sleeve extending longitudinally from the proximal end to the distal end.

12. The device of claim 11 wherein the proximal end of the inner sleeve increases in size to limit passage of the inner sleeve through the outer sleeve.

13. The device of claim 11 wherein a cross section of the opening of the inner sleeve along the longitudinal axis is smaller than a cross section of the opening of the outer sleeve along the longitudinal axis.

14. The device of claim 11 further comprising:
a flared end of the inner sleeve located at the proximal end wherein the flared end is sized not to pass through the opening of the outer wall.

15. The device of claim 11 wherein the outer sleeve smoothly transitions to the inner sleeve.

16. The device of claim 1 wherein the inner sleeve is flexible and the outer sleeve is flexible.

17. The device of claim 11 wherein the opening of the outer sleeve is sized to accept the insertion of at least two electrically conductive leads through the outer sleeve from the proximal end to the distal end.

18. A method of inserting a medical component into a patient, the method comprising:
inserting a guide wire into the patient;
placing an opening at a distal end of a dilator catheter on the guide wire, wherein the dilator catheter has an inner sleeve and an outer sleeve, wherein the opening extends through both the inner sleeve and the outer sleeve;
removing the guide wire through the outer sleeve;
positioning the outer sleeve along the guide wire into a desired position in the patient;
removing the inner sleeve from the dilator catheter;
inserting the medical component into an opening extending from a proximal end of the outer sleeve to a distal end of the outer sleeve.

19. The method of claim 18 wherein a proximal end of the inner sleeve increases in size to limit passage of the inner sleeve through the outer sleeve wherein the proximal end of the inner sleeve provides an increased surface area for removing the inner sleeve from the outer sleeve.

20. The method of claim 18 wherein the opening in the outer sleeve extending from the proximal end of the outer sleeve to the distal end of the outer sleeve is large enough to accept insertion of at least two electrically conductive leads.
